(19) United States
(12) Patent Application Publication
(10) Pub. No.: US 2011/0196238 A1
(43) Pub. Date: Aug. 11, 2011

(54) System and Method for Fetal Heart Monitoring Using Ultrasound

(76) Inventors: Nathan A. Jacobson, Oronoco, MN (US); Kale D. Bodily, Oronoco, MN (US)

(21) Appl. No.: 13/021,834

(22) Filed: Feb. 7, 2011

Related U.S. Application Data
(60) Provisional application No. 61/302,014, filed on Feb. 5, 2010.

(51) Int. Cl. A61B 8/00

(52) U.S. Cl. 600/459

(57) ABSTRACT

An ultrasound adapter system for performing ultrasound scanning on a target area of a subject over an extended period of time includes a flexible membrane formed to define an interior enclosure and an exterior. The system also includes an ultrasound gel arranged in the interior of the flexible membrane and at least one opening formed in the flexible membrane to provide access to the interior and restricted from allowing the ultrasound gel to pass through the at least one opening. The system further includes a mounting system attached to the exterior of the flexible membrane proximate to the at least one opening. The mounting system includes a mounting adapter substantially surrounding the at least one opening and a set of adapters extending from the ring and configured to engage an ultrasound probe extending through the ring and the at least one opening and into the ultrasound gel and hold the ultrasound in a substantially fixed position in the ultrasound gel relative to the mounting system.

### CROSS-REFERENCE TO RELATED APPLICATIONS

This application is based on U.S. Provisional Patent Application Ser. No. 61/302,014 filed on Feb. 5, 2010, and entitled “SYSTEM AND METHOD FOR FETAL HEART RATE MONITORING USING ULTRASOUND.”

### BACKGROUND OF THE INVENTION

Fetal heart rate and heart beat monitoring is an important clinical parameter. For example, fetal heart parameters, including heart rate, are monitored to help identify and prevent complications during childbirth. The inability to detect fetal heart tones prior to the crowing of a baby's head has lead to an increase in operative vaginal deliveries, for example, vacuum and forceps procedures, that allow quicker delivery of the baby. These operative deliveries can lead to increased morbidity for both baby and mother.

The most commonly used device for monitoring of fetal heart parameters is the fetal heart rate transducer. Current obstetric fetal heart rate transducers are hard, static discs that frequently slide down the laboring maternal abdomen and make it difficult to detect fetal heart rate when the baby is more than plus-two station, that is, when the fetal head and shoulders drop into the pelvis. As a result, the fetal ultrasound monitor is adjusted frequently to account for the constant descent of the baby and movements of the mother. Heart tones are frequently lost as the baby's head and shoulders descend under the maternal pubic bone, often requiring a trained nurse to stop performing other important tasks and manually locate and track the fetal heart tones.

Alternate methods for acquiring fetal heart rate data use internal monitors such as the fetal scalp electrodes, which have spiral corkscrews that are screwed into the baby's head. These internal monitoring techniques have significant risks such as trauma, infection, or bleeding. In some cases, fetal heart parameters, including heart rate, are detected with a fetal ultrasound monitor by firmly pressing its transducer head against the abdomen of a delivering mother, delivering ultrasound waves through the mother to the child, and monitoring the response of the ultrasound waves as they are reflected back to the transducer head. However, the use of traditional ultrasound transducers and associated ultrasound systems typically require a physician or dedicated technician to properly position and operate the ultrasound system, particularly, during the complex and ever-changing labor environment.

It would therefore be desirable to have a fetal heart parameter monitoring system that provides improved stability, easy manipulation, and improved efficiency from trained staff during childbirth.

### SUMMARY OF THE INVENTION

The present invention overcomes the drawbacks of previous systems and methods by providing a prepackaged adaptor system that allows an ultrasound probe to be secured to a subject for an extended period of time and during childbirth. The adaptor system is specifically designed to be secured to the laboring woman and position an ultrasound transducer for extended monitoring of fetal heart parameters, without the need for constant intervention by a physician, technician, or other clinician.

In accordance with one aspect of the invention, an ultrasound adapter system for performing ultrasound scanning on a target area of a subject over an extended period of time is disclosed that includes a flexible membrane formed to define an interior enclosure and an exterior. The system also includes an ultrasound gel arranged in the interior of the flexible membrane and at least one opening formed in the flexible membrane to provide access to the interior and restricted from allowing the ultrasound gel to pass through the at least one opening. The system further includes a mounting system attached to the exterior of the flexible membrane proximate to the at least one opening. The mounting system includes a mounting adapter substantially surrounding the at least one opening and a set of adapters extending from the ring and configured to engage an ultrasound probe extending through the ring and the at least one opening and into the ultrasound gel and hold the ultrasound in a substantially fixed position in the ultrasound gel relative to the mounting system.

In accordance with another aspect of the invention, an ultrasound system for performing ultrasound scanning on a target area of a subject over an extended period of time is disclosed that includes a flexible membrane having arranged therein an ultrasound gel. The system also includes a restricted opening formed in the flexible membrane on a first side and an adapter system attached to the flexible membrane proximate to restricted opening. The system further includes an ultrasound probe configured to engage the adapter system and, when engaged therewith, penetrate the restricted opening formed in the flexible membrane to be arranged, at least partially, within the ultrasound gel. The adapter system and ultrasound probe are configured to form a rotational axis extending there-through to allow the ultrasound probe to rotate about the rotational axis while arranged at least partially within the ultrasound gel and be fixed in a desired position about the rotational axis to perform ultrasound scanning on the target area of the subject over the extended period of time.

Various other features of the present invention will be made apparent from the following detailed description and the drawings.

### BRIEF DESCRIPTION OF THE DRAWINGS

FIG. 1 is a side elevational view of an adaptor system for ultrasonic fetal heart parameter monitoring in accordance with the present invention.

FIG. 2 is a plan view of the adaptor system of FIG. 1.

FIG. 3 is a side view of an ultrasound probe and adapter system in accordance with the present invention.

### DETAILED DESCRIPTION OF THE INVENTION

The present invention is an ultrasound system that includes an adjustable ultrasound probe that can be fixed to, and easily manipulated within, a gel/fluid base, which is fastened to a maternal abdomen. It allows the detection of heart parameters, including heart rate, during childbirth and decreases the need for invasive internal monitors and operative vaginal deliveries.

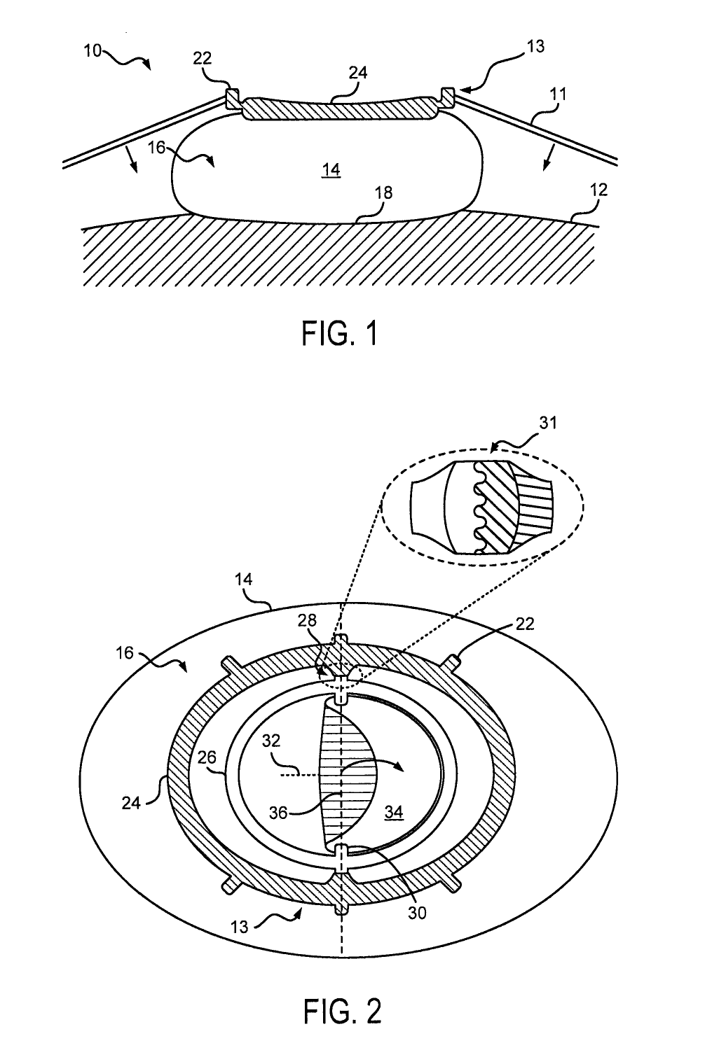

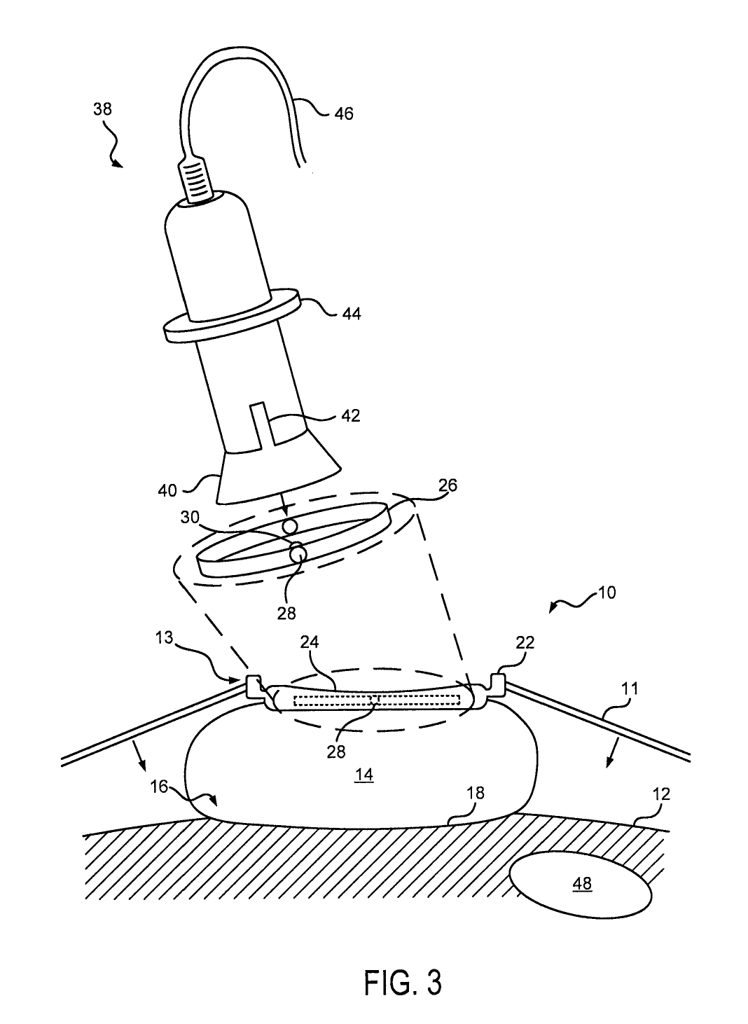

Referring to FIG. 1, the present invention includes a base unit 10 that is fastened using, for example, a belt 11 to a subject 12 that attaches to a mounting system 13 that includes belt hooks 22. The base unit 10, belt 11, and belt hooks 22, are designed to advantageously place a slight downward pressure on the base unit 10, stabilizing the base unit 10 and, through the mounting system 13, allows an adjustable ultrasound probe (such as illustrated in FIG. 3) to be attached over a target area of the subject 12.

It is known that large impedance differences at material boundaries cause the reflection of ultrasound waves and lower ultrasound monitor performance. Large impedance differences at material boundaries are often prevalent if there is a layer of air between an ultrasound transducer head and the subject being scanned. In traditional ultrasound systems, a gel is typically applied directly to the subject to serve as an interfacing medium between an ultrasound transducer and the subject. However, in the childbirth environment, gel applied directly to the woman in labor, overtime, tends to become dispersed. Thus, physician or other clinician must regularly reapply the gel, else risk a reduced ability to accurately monitor conditions using the ultrasound system.

To overcome this problem with the use of traditional ultrasound systems, the present invention includes a pre-packaged, substantially spheroidal, flexible membrane 14, such as the illustrated bag, defining an exterior and an interior containing a scanning gel 16, which, as will be described, a transducer head may be placed within. The bottom of the flexible membrane 14, that is, the area in direct contact with the subject, may include a perforated membrane 18 that allows the scanning gel 16 to leak onto the surface of the subject 12 when subjected to the downward pressure described above. This provides reduced impedance at the material interfaces and improved scanning performance. It is contemplated that the perforated membrane is covered by a protective pull-tab before use to prevent unwanted leakage of the scanning gel 16. It is further contemplated that the flexible membrane 14 is made of a conducive soft plastic material, for example, soft-pliable latex. Moreover, the texture of the bag material may vary across the surface of the bag to provide increased friction and improved grip.

Referring now to FIG. 2, the mounting system 13 includes an outer station-ring 24. The station ring 24 is designed to support the belt adaptors 22, for example, belt hooks or snaps, that, as described above, that attach to the belt 11. It is contemplated that the station ring 24 may be directly attached to the flexible membrane 14. The mounting system 13 also includes an inner tilt-ring 26 is connected to the station ring 24 via joints 28 that allow rotation about the central axis 36 extending between the two joints 28, thus allowing rotation along a longitudinal axis of the subject 12. It is contemplated that these joints include detent or face gear couplings 31 that allow rotation if sufficient force is applied, but otherwise remain locked at a set angle. The inner tilt-ring 26 further includes base locks 30 that, as will be described, fasten to and secure an ultrasound probe to the base unit 10 for proper positioning of the ultrasound probe, despite dynamic environments, and without the need for constant clinician oversight or intervention.

Still referring to FIG. 2, the surface of the flexible membrane 14 lying within the inner diameter of the tilt-ring 26 includes fenestrations 32 that allow an ultrasound transducer, which has been passed through the inner radius of the tilt-ring 26, to puncture portions of the flexible membrane 14, allowing the transducer head to be submersed in the gel 16. The region of the flexible membrane 14 having fenestrations may be covered with a removable pull-tab 34 to prevent unwanted leakage of gel prior to use.

Referring to FIG. 3, an ultrasound probe, indicated generally at 38, is provided that includes a transducer head 40 that is capable of producing and receiving ultrasound waves and a cable 46 that conveys acquired ultrasound information to a computer (not shown) that produces diagnostic information, for example, heart rate, from the acquired ultrasound signal. The ultrasound probe 38 may be designed to be mounted to the base unit 10 through the mounting system 13, which is secured to the subject 12 with the belt 11 as described above. However, it is also contemplated that the ultrasound probe 38 may be an ultrasound probe, which is already on the market, that is fitted with an universal adaptor set, thereby allowing the probe to be used in accordance with the present invention. During the mounting process the probe 38 is passed through the inner tilt-ring 26 and disposed so that the transducer head 40 is then inserted within the flexible membrane 14, thereby puncturing the area of the flexible membrane 14 having fenestrations 32, such as illustrated in FIG. 2. The transducer head 40 is freely submerged within the flexible membrane 14, but, preferably, does not directly touch the bottom of the flexible membrane 14. This allows free movement of the transducer head 40 by rotation at joints 28. As mentioned above, the downward pressure provided by the belt 11, which is attached to the outer stationary ring 24, allows the gel 16 to leak through the perforated membrane 18 onto the subject 12, providing an improved interface for performing ultrasound.

The probe 38 is further secured within the base by the transducer groove locks 42, which attach to the base locks 30. The ultrasound probe 38 may include a flange 44 that is designed to be moved down the probe body and fitted securely to the tilt-ring 26 to prevent the leakage of the scanning gel 16 about the probe 38.

Referring to FIGS. 2 and 3, when inserted into the base unit 10, the adjustable transducer head may rotate about the axis 36 by rotating on the hinges 28. Both the inner tilt ring 26 and the outer station ring 24 are attached to the flexible membrane 14. However, the outer stationary ring 24 is firmly attached by the belt system 11 while the portion of the flexible membrane 14 between the inner and outer ring includes extra material, “slack,” to allow for rotation of the inner tilt ring 26 out of the plane of the outer station ring 24, while not allowing any gel leakage due to the slight downward pressure produced by the belt hook system.

As shown in FIG. 3, following mounting, the ultrasound probe may perform ultrasound scanning for an extended duration of time while secured in a fixed location on a subject, for example, with respect to a particular region of interest 48, such as just under the pubic bone of a woman in labor. The rotating joints 28 allow the ultrasound probe 38 to be angled and repositioned within the base 10, allowing the ultrasound system to be rotated in order to track and monitor a moving feature, for example, fetal heart rate during childbirth, or compensate for subject movement. While the present invention is primarily intended for monitoring fetal heart rate during childbirth, it is contemplated that it could be used for other situations, for example, physical therapy or ultrasound guided injections, in which an ultrasound scan of a fixed location in a subject is performed over an extended period of time.

The present invention has been described in accordance with the embodiments shown, and one of ordinary skill in the art will readily recognize that there could be variations to the embodiments, and any variations would be within the spirit and scope of the present invention. Accordingly, many modifications may be made by one of ordinary skill in the art without departing from the spirit and scope of the appended claims.

1. An ultrasound adapter system for performing ultrasound scanning on a target area of a subject over an extended period of time, the ultrasound adapter system comprising:
a flexible membrane formed to define an interior enclosure and an exterior;
an ultrasound gel arranged in the interior of the flexible membrane;
at least one opening formed in the flexible membrane to provide access to the interior and restricted from allowing the ultrasound gel to pass through the at least one opening;
a mounting system attached to the exterior of the flexible membrane proximate to the at least one opening and comprising:
a mounting adapter substantially surrounding the at least one opening; and
a set of adapters extending from the ring and configured to engage an ultrasound probe extending through the ring and the at least one opening and into the ultrasound gel and hold the ultrasound in a substantially fixed position in the ultrasound gel relative to the mounting system.

2. The system of claim 1 wherein the at least one opening is restricted from opening by at least one of a perforated membrane and fenestrations.

3. The system of claim 1 wherein the mounting adapter includes a station-ring extending about and substantially coaxially with a tilt-ring, wherein the tilt-ring is configured to adjust through a plurality of positions relative to the flexible membrane to allow adjustment of the ultrasound probe when engaged with the set of adapters.

4. The system of claim 3 wherein the mounting adapter further includes rotational joints extending to connect the station-ring to the tilt-ring to form a rotational axis there-through to allow the ultrasound probe engaged with the tilt-ring through the set of adapters to rotate about the rotational axis.

5. The system of claim 4 wherein the rotational joints include face-gear couplings that allow the ultrasound probe to rotate about the rotational axis upon application of a force sufficient to overcome a bias of the face-gear couplings.

6. The system of claim 1 further comprising a removable covering the at least one opening to prevent undesired leaking of the ultrasound gel contained within the flexible membrane.

7. The system of claim 1 wherein an exterior surface of the flexible membrane includes a texture configured to increase frictional forces when engaged with the subject.

8. An ultrasound system for performing ultrasound scanning on a target area of a subject over an extended period of time, the ultrasound system comprising:
a flexible membrane having arranged therein an ultrasound gel;
a restricted opening formed in the flexible membrane on a first side;
an adapter system attached to the flexible membrane proximate to restricted opening;
an ultrasound probe configured to engage the adapter system and, when engaged therewith, penetrate the restricted opening formed in the flexible membrane to be arranged, at least partially, within the ultrasound gel; and
wherein the adapter system and ultrasound probe are configured to form a rotational axis extending there-through to allow the ultrasound probe to rotate about the rotational axis while arranged at least partially within the ultrasound gel and be fixed in a desired position about the rotational axis to perform ultrasound scanning on the target area of the subject over the extended period of time.

9. The system of claim 8 wherein the adapter system includes a tilt-ring and a station-ring extending about and substantially coaxially to the tilt-ring and, wherein an interface of the station-ring and the tilt-ring form the rotational axis through connection between the station-ring and the tilt-ring.

10. The system of claim 1 wherein the ultrasound probe further includes a flange configured to attach to the adapter system to form a seal between the ultrasound probe and at least one of the adapter system and the flexible membrane to restrict the scanning gel from leaking from the flexible membrane through the restricted opening.

11. The system of claim 1 further comprising another restricted opening formed in the flexible membrane and arranged substantially opposite the restricted opening to allow the ultrasound gel to be disposed on the subject.
